(19) United States
(12) Patent Application Publication
(10) Pub. No.: US 2022/0240605 A1
(43) Pub. Date: Aug. 4, 2022

(54) Face Mask Having a Combined Biocidal and Electrostatic Treatment

(71) Applicant: BioSerenity, Paris (FR)

(72) Inventors: Pierre-Yves Frouin, Atlanta, GA (US); Bernard Gerard Emmanuel Martel, Nieppe (FR); Nicholas Jean Yves Blanchemain, Merville (FR)

(73) Assignee: BioSerenity, Paris (FR)

(21) Appl. No.: 17/162,276

(22) Filed: Jan. 29, 2021

(51) Int. Cl. A41D 13/11; A41D 31/30; A41D 31/14; A62B 23/02

(52) U.S. Cl. CPC A41D 13/1192; A62B 23/025; A41D 31/145; A41D 31/305

(57) ABSTRACT

A face mask having mechanical, chemical, and electrostatic barriers to the movement of a material into and out of a face mask are described herein. Various examples of the face mask described herein use electrostatic charges to capture and, in some examples, inactivate viruses, germs, fungi, or bacteria (or other material). The use of a biocidal agent combined with electrostatic charges provides an additional measure for capturing and inactivating at least a portion of pathogen(ic) microorganisms.

### BACKGROUND

Face masks, such as surgical masks, N95 respirators, KN95 respirators, a bandana-type face mask, a pull-up face mask, a gaiter mask, cloth masks, surgical mask, European standard FFP2 Mask, and the like are often used to reduce the probability of the introduction of a particulate or biological agent (such as a virus or bacteria) into the mouth and/or nose of the wearer of the mask. Respiratory face masks are a form of personal protective equipment (“PPE”), or medical device in general, worn on the face or head that are intended to cover at least, if not all, of the mouth and nose of the wearer and protect the wearer from particulate, dirt, dust, chemical agents or vapors, viruses, bacteria, and/or germs in general. In some examples, the respiratory face masks may be disposable; however, some respiratory face masks can be cleaned or sterilized for reuse. Likewise, face masks are often used to reduce the probability of the communication of a particulate or pathogen(ic) microorganism (such as a virus, a fungi or a bacteria) from the mouth and/or nose of the wearer of the mask to another individual or on surfaces in the proximity of the wearer of the mask.

It is with these and other concerns that an improved face mask is described herein.

### BRIEF DESCRIPTION OF THE DRAWINGS

The detailed description is set forth with reference to the accompanying figures. The use of the same reference numbers in different figures indicates similar or identical items or features.

FIG. 1 illustrates filtration mechanisms that may be used to block or capture particles or organisms, in accordance with some example of the present disclosure.

FIG. 2 is an illustration of an example respiratory face mask, in accordance with some examples of the present disclosure.

FIG. 3 is an illustration showing stages of capture and destruction of a pathogen microorganism, in accordance with some examples of the present disclosure.

FIG. 4 illustrates the face mask using zones, in accordance with some examples of the present disclosure.

FIG. 5 is an illustration showing a face mask with multiple layers, in accordance with some examples of the present disclosure.

FIG. 6 is an example process for manufacturing a face mask, in accordance with some examples of the present disclosure.

### DETAILED DESCRIPTION

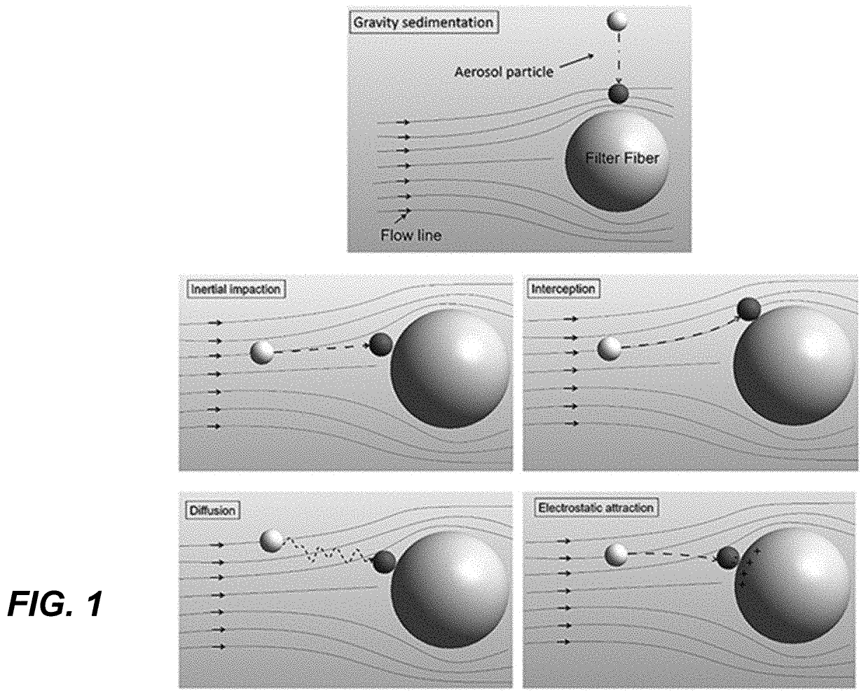

Examples of the present disclosure can comprise a face mask having a biocidal agent and electrostatic charges embedded or located on a surface of a face mask. In some examples, the electrostatic charges are used to electrostatically attract pathogen microorganisms to biocidal agents located on fibers of a face mask. In conventional, non-electrostatically charged masks, the ability of the mask to capture pathogen microorganisms is limited to certain flow mechanisms, such as some of those illustrated in FIG. 1. Examples of the present disclosure, a layer of fabric having combined electrostatic and biocidal properties, may be used in other, non-mask and/or non-filtering applications, such as gowns, coverings, and the like. The presently disclosed subject matter is not limited to a use on masks. Examples of various aspects, including combinations thereof, may include the following aspects. Other aspects are disclosed herein, as these aspects are merely examples.

In a first aspect, a face mask is described. The face mask includes a plurality of biocidal agents deposited on a first surface or a second surface of a material used to construct the face mask; a plurality of electrostatic charges deposited using a high voltage source on the first surface or the second surface of the material used to construct the face mask; and wherein the plurality of electrostatic charges increases a probability that a pathogen microorganism is captured by the first surface or the second surface and the plurality of biocidal agents kill or render inert the pathogen microorganisms that are captured. Other aspects of the face mask of the first aspect include, but are not limited to, wherein the plurality of electrostatic charges is configured to increase an efficacy of a material used to construct the face mask to capture the pathogen microorganism, wherein the face mask comprises an N95 respirator, a KN95 respirator, a bandana-type face mask, a pull-up face mask, a gaiter mask, a cloth mask, a European standard FFP2 Mask, an Australian P2 Mask, Korean 1^{st} class Mask, Japanese DS Mask, and a surgical mask, wherein the plurality of biocidal agents comprises benzalkonium chloride complexed in cyclodextrin cavity bonded on fibers of the material of the face mask, wherein the plurality of biocidal agents comprises antimicrobials, viricidals, antibiotics, antibacterials, antivirals, antifungals, antiprotozoals, and antiparasites, wherein the biocidal agents comprise Dimethyldioctadecylammonium chloride, wherein the biocidal agents comprise alkyldimethylbenzylammonium chloride, wherein the biocidal agents comprise Benzethonium chloride, wherein the face mask comprises a plurality of zones, wherein a concentration of the plurality of the biocidal agents and a concentration of the plurality of electrostatic charges varies between zones of the plurality of zones, and wherein a first zone of a plurality of zones inline with a direction of respirated air from a mouth of a wearer of the face mask has a higher concentration of either of the plurality of the biocidal agents or the plurality of the electrostatic charges than a second zone of a plurality of zones proximate to a side of a face of the wearer of the face mask.

A second aspect of the present disclosure includes a method of manufacturing a face mask. The method includes providing a first layer having a first type of polypropylene nonwoven such as, but not limited to, a meltblown or spunbond polypropylene nonwoven; providing a second layer having a second type of spun bond polypropylene; applying a plurality of biocidal agents to the second layer; applying a high voltage field to the second layer to deposit a plurality of electrostatic charges on the second layer; and bonding the second layer to the first layer, wherein the plurality of electrostatic charges increases a probability that a pathogen microorganism is captured by a surface of the second layer and the plurality of biocidal agents kill or render inert the pathogen microorganisms that are captured at the surface of the second layer. The method of the second aspect further includes, in various combinations: providing a third layer having a first type of spun bond polypropylenes applying a plurality of biocidal agents to the third layer, applying a high voltage field to the third layer to deposit a plurality of electrostatic charges on the third layer, and bonding the third layer to the second layer, wherein the plurality of electrostatic charges increases a probability that a pathogen microorganism is captured by a surface of the third layer and the plurality of biocidal agents kill or render inert a portion of the pathogen microorganisms that are captured at the surface of the third layer; providing a third layer having a third type of spun bond polypropylene, applying a plurality of biocidal agents to the third layer, and bonding the third layer to the second layer; providing a third layer having a third type of spun bond polypropylene, applying a high voltage field to the third layer to deposit a plurality of electrostatic charges on the third layer, and bonding the third layer to the second layer; providing a fourth layer having a fourth type of spun bond polypropylene, and bonding the fourth layer to the third layer; wherein the face mask comprises an N95 respirator, a KN95 respirator, a bandana-type face mask, a pull-up face mask, a gaiter mask, a cloth mask, a European standard FFP2 Mask, and a surgical mask; wherein the plurality of biocidal agent comprises benzalkonium chloride complexed in cyclodextrin cavity bonded on fibers of a material of the face mask; wherein the plurality of biocidal agents comprises fungicides, antimicrobials, viricidals, antibiotics, antibacterials, antivirals, antifungals, antiprotozoals, antiparasites, and combinations thereof; wherein the biocidal agents comprise at least one of Dimethyldioctadecylammonium chloride, alkyldimethylbenzylammonium chloride, and Benzethonium chloride; wherein applying a plurality of the biocidal agents to the second layer comprises identifying a first zone for a first density of the plurality of biocidal agents, identifying a second zone for a second density of the plurality of biocidal agents, and depositing the plurality of biocidal agents in the first zone at the first density and the second zone at the second density; wherein applying a high voltage field to the second layer comprises identifying a first zone for a first density of the plurality of electrostatic charges, and identifying a second zone for a second density of the plurality of electrostatic charges, and applying the high voltage field at a first potential to deposit the plurality of electrostatic charges in the first zone at the first density and at a second potential to deposit the plurality of electrostatic charges in the second zone at the second density.

A third aspect of the present disclosure includes a face mask. The face mask includes a first layer having a first type of spun bond polypropylene, a second layer having a second type of spun bond polypropylene, a plurality of biocidal agents on the second layer, a plurality of electrostatic charges on the second layer deposited using a high voltage field applied to the second layer to deposit, and the second layer bonded to the first layer, wherein the plurality of electrostatic charges increases a probability that a pathogen microorganism is captured by a surface of the second layer and the plurality of biocidal agents kill or render inert the pathogen microorganisms that are captured at the surface of the second layer. Other aspects may include combinations of a third layer having a first type of spun bond polypropylene, a plurality of biocidal agents on the third layer, a plurality of electrostatic charges on the third layer deposited using a high voltage field applied to the third layer to deposit, and the third layer bonded to the second layer, wherein the plurality of electrostatic charges increases a probability that a pathogen microorganism is captured by a surface of the third layer and the plurality of biocidal agents kill or render inert a portion of the pathogen microorganisms that are captured at the surface of the third layer. Further aspects may include a third layer having a third type of spun bond polypropylene; a plurality of biocidal agents to the third layer; and the third layer bonded to the second layer. Still further aspects may include a third layer having a third type of spun bond polypropylene; a plurality of electrostatic charges on the third layer deposited by applying a high voltage field to the third layer; and the third layer bonded to the second layer. Other additional aspects may include a fourth layer having a fourth type of spun bond polypropylene; and the fourth layer bonded to the third layer. Further additional aspects may include wherein the face mask comprises an N95 respirator, a KN95 respirator, a bandana-type face mask, a pull-up face mask, a gaiter mask, a cloth mask, a European standard FFP2 Mask, and a surgical mask, wherein the plurality of biocidal agent comprises benzalkonium chloride complexed in cyclodextrin cavity bonded on fibers of a material of the face mask, wherein the plurality of biocidal agents comprises fungicides, antimicrobials, viricidals, antibiotics, antibacterials, antivirals, antifungals, antiprotozoals, antiparasites, and combinations thereof, wherein the plurality of biocidal agents comprises at least one of Dimethyldioctadecylammonium chloride, alkyldimethylbenzylammonium chloride, and Benzethonium chloride.

A fourth aspect of the present disclosure includes various combinations of a fabric. In some example, the layer of fabric includes a first layer having a first type of spun bond polypropylene; a second layer having a second type of spun bond polypropylene; a plurality of biocidal agents on the second layer; a plurality of electrostatic charges on the second layer deposited using a high voltage field applied to the second layer to deposit; and the second layer bonded to the first layer, wherein the plurality of electrostatic charges increases a probability that a pathogen microorganism is captured by a surface of the second layer and the plurality of biocidal agents kill or render inert the pathogen microorganisms that are captured at the surface of the second layer. Other aspects may include a third layer having a first type of spun bond polypropylene; a plurality of biocidal agents on the third layer; a plurality of electrostatic charges on the third layer deposited using a high voltage field applied to the third layer to deposit; and the third layer bonded to the second layer, wherein the plurality of electrostatic charges increases a probability that a pathogen microorganism is captured by a surface of the third layer and the plurality of biocidal agents kill or render inert a portion of the pathogen microorganisms that are captured at the surface of the third layer. Still further aspects may include a third layer having a third type of spun bond polypropylene; a plurality of biocidal agents to the third layer; and the third layer bonded to the second layer. Additional aspects may include a third layer having a third type of spun bond polypropylene; a plurality of electrostatic charges on the third layer deposited by applying a high voltage field to the third layer; and the third layer bonded to the second layer. Additional further aspects may include a fourth layer having a fourth type of spun bond polypropylene; and the fourth layer bonded to the third layer. Still further aspects may include wherein the face mask comprises an N95 respirator, a KN95 respirator, a bandana-type face mask, a pull-up face mask, a gaiter mask, a cloth mask, a European standard FFP2 Mask, and a surgical mask; wherein the plurality of biocidal agent comprises benzalkonium chloride complexed in cyclodextrin cavity bonded on fibers of a material of the face mask; wherein the plurality of biocidal agents comprises fungicides, antimicrobials, viricidals, antibiotics, antibacterials, antivirals, antifungals, antiprotozoals, antiparasites, and combinations thereof; wherein the plurality of biocidal agents comprises at least one of Dimethyldioctadecylammonium chloride, alkyldimethylbenzylammonium chloride, and Benzethonium chloride.

FIG. 1 illustrates filtration mechanisms that may be used to block or capture particles or organisms. In some examples, various filtration mechanisms in a textile medium may include, but are not limited to, gravity sedimentation, inertial impaction, interception, and diffusion. Gravity sedimentation, inertial impaction, interception, and diffusion intrinsically depend on the air flow, the particles size and the structuration of the textile (thickness, specific area, density, fibers diameters). The probability that a pathogen microorganism is trapped or captured by a fiber of a mask can be enhanced using electrostatic attraction. By providing an additional capture mechanism, electrostatic attraction can enhance the filtration characteristics of a face mask by increasing the probability that a pathogen microorganism is captured by a fiber. Electrostatic charges present on the fibers can polarize aerosols traveling in the air flow (solid or liquid micrometric or nanometric particles) and intercept them by attractive electrostatic forces.

However, the mere increasing the capture of a pathogen microorganism may not be enough. For example, even if the pathogen microorganism is captured on a fiber of a face mask, the pathogen microorganism may still be alive and viable. This means that if the face mask is thrown away or disposed of, the face mask may be a danger because of the sequestration of a potentially significant number of viable and alive pathogen microorganisms on the fibers of the face mask, essentially leading to a localized “hot spot” of activity. Further, the wearer of the mask, either through daily activities or when taking off the mask, may impart enough movement on the face mask that once captured pathogen microorganisms are released back into the vicinity of the wearer of the face mask.

To alleviate these and other issues, examples of the presently disclosed subject matter use electrostatic charges to increase the probability that a pathogen microorganism is captured by a fiber of the face mask and biocidal agents on those fibers to kill or render inert the captured pathogen microorganisms. As used herein, a biocidal agent is a chemical or other material that act, and preferably destroy, at least a portion of an organic substance, such as a bacteria, fungi, or virus. As used herein, a “biocidal agent” includes, but is not limited to, fungicides, herbicides, insecticides, algicides, molluscicides, miticides, rodenticides, slimicides, germicides, antimicrobials, viricidals, antibiotics, antibacterials, antivirals, antifungals, and antiparasites, and the like (collectively and generically referred to herein as “biocidal agents”). The term “antibacterial” denotes any effect of inhibiting the growth of bacteria (bacteriostatic) or destroying said bacteria (bactericides), or fungi (fungicides). The term antiviral means any effect of inhibiting virus growth (virostatic) or destroying said viruses (virucidal). Antifungal means any effect of inhibiting the growth of fungi (fungistatic) or destroying said fungi (fungicide).

However, using conventional technologies, it has been difficult, or impossible, to place electrostatic charges proximate to biocidal agents. A reason for this is that conventional antiviral masks are typically made of a layer of cellulosic fabric loaded by impregnation, by spraying or by chemical grafting of some mineral or organic antiviral agents. In case of a face mask, it is important that the biocide compound is bound to the fibers in order to avoid leaching of biocide from the mask toward skin, mouth and nose of the wearer. Therefore, the textile must have retention properties toward the biocide in order to prevent potential toxic or allergen reaction caused by the biocide. Therefore, the biocide can be chemically bound to the fiber by covalent bonds, or physically bound by Van der Waals forces, hydrogen bonds, acid-base or ionic interactions. Cellulosic fabric has historically been shown to be readily functionalized with biocidal agents because of the good chemical reactivity of the cellulosic fabric, and high adsorption capacity of aqueous solutions containing biocidal agents (organic or mineral) and high retention of the biocide agents through physical bonding. However, such cellulosic fibers often cannot accumulate a relatively high electrostatic charge, and even in some examples in which a charge is accumulated, the charges typically rapidly decay. In some examples, the moisture found in respiratory air flow can be absorbed by the cellulosic fibers, contributing to the decay of the any remaining electrostatic charge.

The result of this is that the benefit of the electrostatic treatment of antiviral cellulosic fibers is poor and not sustainable. Therefore, biocidal masks based on cellulosic fibers often include an additional layer of synthetic fibers, mostly a melt blown nonwoven made of polypropylene (PP) of polyethylene terephthalate (PET), that bring the high filtration performance to such masks. Conventional biocidal masks that use melt blown or spun bond PP or PET, as well as other suitable “synthetic fibers,” while being more capable of accumulating a relatively high electrostatic charge as compared to cellulosic fabric or fibers, do not present high retention properties with biocidal agents due to their hydrophobicity and their low ability to interact with biocides through physical interactions. Thus, in conventional masks based on antiviral cellulosic fibers, the combination of filtration mechanisms, such as those illustrated in FIG. 1, and electrostatic charges has been difficult or ineffective.

A face mask according to various examples disclosed herein overcome the limitations and deficiencies found in conventional systems. In order to do this, in some examples, a melt blown PP nonwoven is first functionalized with a quaternary ammonium coating the fibers accordingly with the process reported in French Patent FR2984176A1. One example includes one alkyl group composed of 8 to 16 carbons and one alkyl group composed of 8 to 16 carbons, such as Dimethyldioctadecylammonium chloride (DODMAC). Another example includes one alkyl group composed of 8 to 16 carbons and one phenyl group or one benzyl or one alkylphenyl group, such as alkyldimethylbenzylammonium chloride (ADBAC). In a still further example, the coating may include one benzyl group and one alkylephenoxyethoxyethyl group, such as Benzethonium chloride. The process reported by patent FR2984176A1 describes the surface modification of synthetic fibers by coating with a polymer of cyclodextrin. Cyclodextrin is a torus shaped cyclic oligosaccharide that forms a hydrophobic cavity inside which alkyl chains or aromatic groups of a biocide agent can enter and form stable and reversible “inclusion complexes”. Patent FR2984176A1 describes that textiles modified with such cyclodextrins polymers present high affinity and retention properties toward biocide agents in particular those of the family of quaternary ammoniums. So, applying the process described in patent FR2984176A1 to synthetic fabric (e.g. meltblown PP nonwoven) provides antimicrobial fibers coated with a quaternary ammonium-cyclodextrin complex. Such strategy can be adopted in order to bind the biocide agent to the fiber and thereby prevent the diffusion of the biocide agent from the mask toward the skin, mouth, nose of the wearer, preventing toxic or allergenic risks.

The synthetic nature of PP biocidal fibers allows for the sustainment of the electrostatic charges, while the quaternary ammonium coating provides for biocidal properties. An advantage of this double functionality, i.e. the combination of the electrostatic charges and the biocidal agent on the same textile layer composing the mask is: 1) the electrostatic charges attract the pathogen microorganism or an aerosol vectorizing the pathogen microorganism on the fiber, and 2) the pathogen microorganism enters in contact with the quaternary ammonium present on the same fiber and is can readily be killed. The antiviral agent combined with the electrostatic charges on the same fibers can provide for a synergetic effect that results in a higher efficiency than an assembly of two distinct antiviral and electrostatic textile layers. Further, destroying at least a portion of pathogen microorganisms captured in the mask decreases the probability that these organics escape during removal or handling of the mask, or after the used mask is discarded and become new sources of contamination. In some examples, destroying or killing a pathogen microorganism captured in the mask reduces or eliminates the potential of the discarded well-worn mask to be a hazardous waste.

As used herein, an “electrostatic property” is an electrostatic charge placed on one or more locations of the face mask. The charges are designed to interact with and collect electrically charged or polarized materials. In some examples, the charge itself may polarize small particles that may carry a virus/bacteria. The particles may thereafter be attracted to and captured by electrostatic forces. In other examples, a particle carrying a pathogen microorganism may be intrinsically charged or polarized, such as water droplets, whereby the particle is attracted to and captured by electrostatic forces. The electrostatic charges on the textile filtering medium provide various advantages. For example, the electrostatic charges can work in concert with the material of the face mask to prevent the travel of an unwanted pathogen microorganism through the face mask. In this matter, an increase of electrical charges can essentially increase the effectiveness of the face mask material.

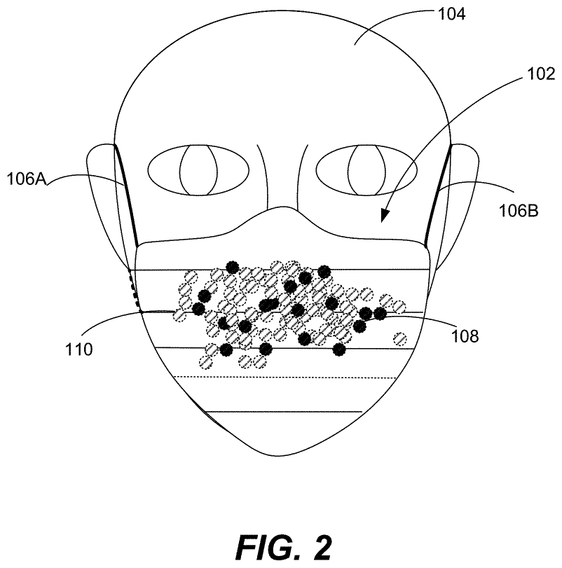

FIG. 2 is an illustration of an example respiratory face mask 102, in accordance with some examples of the present disclosure. The face mask 102 may be an N95 respirator, a KN95 respirators, a bandana-type face mask, a pull-up face mask, a gaiter mask, a cloth mask, an Australian P2 Mask, Korean 1^{st} class Mask, Japanese DS Mask, a surgical mask, a European standard FFP2 Mask, and the like. The presently disclosed subject matter is not limited to any particular type of face mask 102, and, may include face masks that are not designed to allow for respirated air to travel through the face mask. The respiratory face mask 102 is designed to be worn over the face of a user 104. The respiratory face mask 102 is removably secured to the face of the user 104 through the use of straps 106A and 106B, which may be placed over ears or other securement mechanisms. The presently disclosed subject matter is not limited to any particular way in which the respiratory face mask 102 is secured to the face of the user 104.

Illustrated in FIG. 2 are biocidal agents 108, illustrated in FIG. 2 as solid, black dots. These biocidal agents 108 may be present on the inner surface of the face mask 102 facing the user 104, the outer surface of the face mask 102 facing away from the user 104, or inside some areas of the pores of the face mask 102, or combinations thereof. The biocidal agents 108 may be placed at targeted locations on the mask using various technologies and techniques, some of which include, but are not limited to, deposition, bounding, and impregnation. The presently disclosed subject matter is not limited to any particular technology. Although various biocidal agents 108 may be used, in some examples, a quaternary ammonium (Quat) may be complexed in cyclodextrin cavity bonded on fibers of the face mask 102. In some examples, the biocidal agents 108, or antimicrobial materials, are prepared by combining a material with a biocide substance. In some examples, there are three mechanisms of action of an antimicrobial surface with the pathogen microorganisms: the anti-adhesive mechanism acting by preventing the pathogen microorganism to adhere and colonize the surface; the release-killing mechanism acting by releasing the antimicrobial agent that diffuses toward the pathogen microorganism; and the contact-killing mechanism acting by killing the pathogen microorganism once it enters in contact with the surface. In the frame of this invention, the later mechanism is preferably involved. The presently disclosed subject matter is not limited to any particular mechanism. Examples of the manner in which the biocidal agents 108 may be affixed on the face mask, as well as other examples of biocidal agents 108, may be found in French Patent FR2984176A1, which is incorporated herein in its entirety as if fully set forth herein.

In some examples, as discussed above, only using the biocidal agents 108 may not provide the desired level of protection for using the face mask 102. For example, the biocidal agents 108 may require a particular period of time in which to act upon the organic material (e.g. a pathogen microorganism). With the volumetric flowrate of respiration from the user 104, the biocidal agents 108 may simply not have enough time to act upon the organic material. Further, even if the biocidal agents 108 begin to act upon the organic material, the volume of respirated air from the user 104 may push or force incompletely destroyed organic material off of the biocidal agents 108 and back into the respirated air stream.

Thus, in some examples of the presently disclosed subject matter, the face mask includes electrostatic charges 110, illustrated in FIG. 2 as hashed dots. These electrostatic charges 110 may be impregnated onto the inner surface of the face mask 102 facing the user 104, the outer surface of the face mask 102 facing away from the user 104, or through pores of the face mask 102, or combinations thereof. The electrostatic charges 110 are positive charges generated by and deposited using a plasma, corona, or other similar technology. In other examples, the electrostatic charges 110 may be created using magnesium salts or other similarly suitable materials incorporated in the textile. As particles with a negative surface charge, such as some aerosols, dust, bacteria, fungi, or viruses, have negative surface charge, enter in close proximity with the positively charge electrostatic charge 110, the particles are “caught” or trapped by the electrostatic charge 110. It should be noted that the presently disclosed subject matter is not limited to the electrostatic charges 110 being positive charges, as negative charges may be used in lieu of, or in conjunction with, positive charges. In a variant, airborne particles that do not present intrinsic electrostatic charge, can be polarized in proximity of the electrostatic fibers and undergo attraction forces leading to their impaction on charged fibers.

In prior uses, the use of electrostatic charges 110 was often used separately from biocidal agents 108. The reason for this is that the application of the electrostatic charges 110 often destroyed most, if not all, of the biocidal agents 108. In various examples of the presently disclosed subject matter, a high voltage field or source, rather than a plasma or corona, is used to induce electrostatic charges onto the surface of the face mask 102. The higher the potential of the high voltage field, the greater the coverage of electrostatic charges 110 may be realized. In some examples, the high voltage field may be applied after the face mask 102 is constructed with the biocidal agents 108, or, during the spin phase in which individual strands of fiber used to create the face mask 102.

Various materials may be used including, but not limited to, textile medias that are woven, knitted, or nonwovens. The presently disclosed subject matter may be used with any suitable fiber and construction method. It should be noted that although the pore 202 is illustrated as being circular, this is merely for purposes of illustration, as the pore 202 may be of various shapes and sizes.

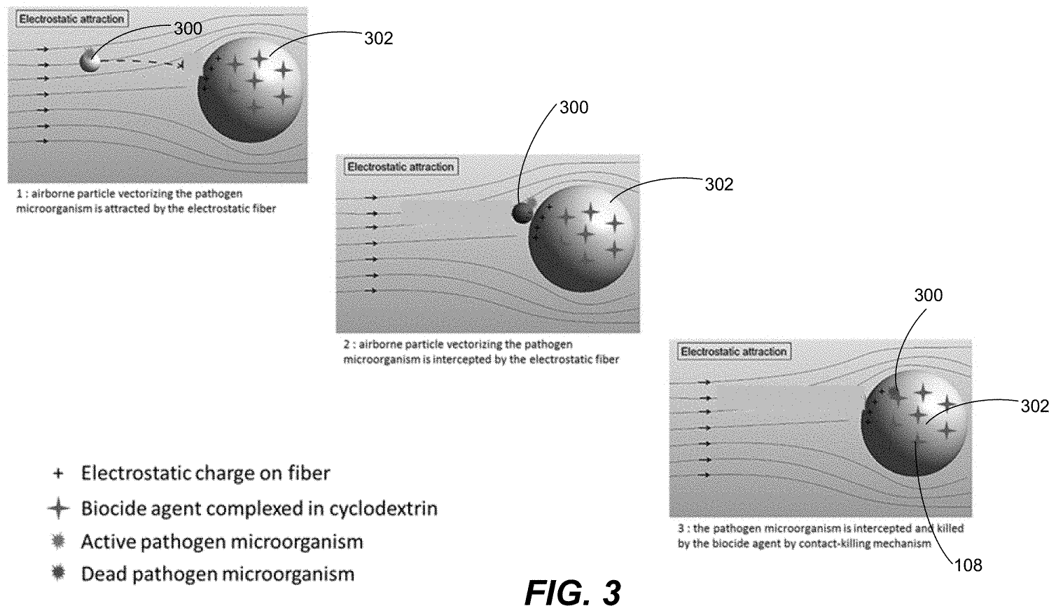

FIG. 3 illustrates the interaction of the electrostatic charges 110 (illustrated as “+” in FIG. 3) on a pathogen microorganism 300. At step 1, the pathogen microorganism 302 is airborne and is vectoring towards a fiber 302 of the face mask 102 of FIG. 1. The pathogen microorganism 302 is attracted to the electrostatic charges on the fiber 302. The electrostatic charges are designed to attract particular organisms, non-organic material, and/or organic material. Thus, when the pathogen microorganism 300 comes in close proximity to one of the electrostatic charges 110, the electrostatic charges 110 pull (electrostatically attract) the material to the electrostatic charges 110 to capture the material. At step 2, the pathogen microorganism 300 is intercepted and captured by the fiber 302 due to the, at least in part, the electrostatic charges on the fiber 302 attracting the pathogen microorganism 300 towards the fiber 302, while the airflow remains unchanged. At step 3, the pathogen microorganism 300 is killed or rendered inert by the biocidal agents 108 on the fiber 302. In some examples, the concerted action of the electrostatic charges to attract the pathogen microorganism 300 and the biocidal agents 108 to destroy the pathogen microorganism 300 may be termed “capture and kill.” In some examples where the biocide agent immobilized on the fiber is a quaternary ammonium compound, the cationic nature of the biocide agent may also interact by electrostatic attraction with the negatively charged membrane of the microorganism (or the viral envelope). Such attractive forces provided by the biocidal cationic agent are independent from the extrinsic electrostatic charge provided by the corona treatment or the magnesium salts but may participate in concert with the later to the attraction and to the capture of the pathogen microorganisms on the fiber.

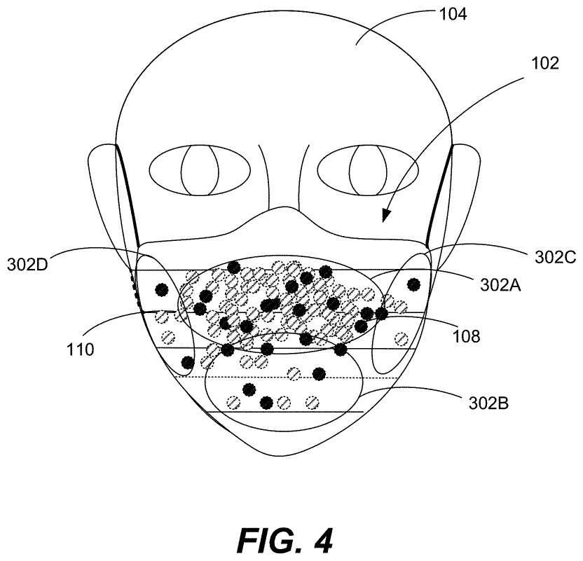

FIG. 4 illustrates the face mask 102 using zones, in accordance with some examples of the presently disclosed subject matter. In some examples, the face mask may have the biocidal and/or electrical charges populated according the expected loading required at a particular point of the face mask. For example, the area of the face mask directly proximate to the mouth of the wearer may have more densely populated area of biocidal agents and/or electrostatic charges as opposed to areas such as above the nose or closer to the outer area of the face mask.

In some examples, it may be desirable to use biocidal agents and electrostatic charges in strategically placed locations to enhance the effect of the biocidal agents and electrostatic charges where needed, while reducing costs by not using as much (or any) biocidal agents and electrostatic charges where likely not needed. Illustrated in FIG. 4 are zones 302A, 302B, 302C, and 302D. Zone 302A is a zone generally in-line with the direction of respirated air leaving the mouth and/or nose of the user 104. Zone 302B is a zone generally below or proximate to the direction of respirated air leaving the mouth and/or nose of the user 104. Zone 302B generally receives less respirated air than zone 302A, but does receive more than zones 302C and 302D, which are located to the outside of the face mask 102.

The zones 302A-D are used to determine the concentration or density of the biocidal agents 108 and/or the electrostatic charges 110. In areas in which there may be a lot of movement of air from and to the mouth and/or nose of the user 104, the concentration/density of the biocidal agents 108 and/or the electrostatic charges 110 may be relatively higher than in areas in which there may be a relatively lower movement of air into and out of, such as the zones 302C and 302D. In areas in which there is a lot of movement, the higher concentration/density can increase the effectiveness of the biocidal agents 108 and/or the electrostatic charges 110, and also increase the longevity of the face mask 102, as the more biocidal agents 108 and/or the electrostatic charges 110 provide for an increased number of locations in which a material may be destroyed by the biocidal agents 108 or captured by the electrostatic charges 110.

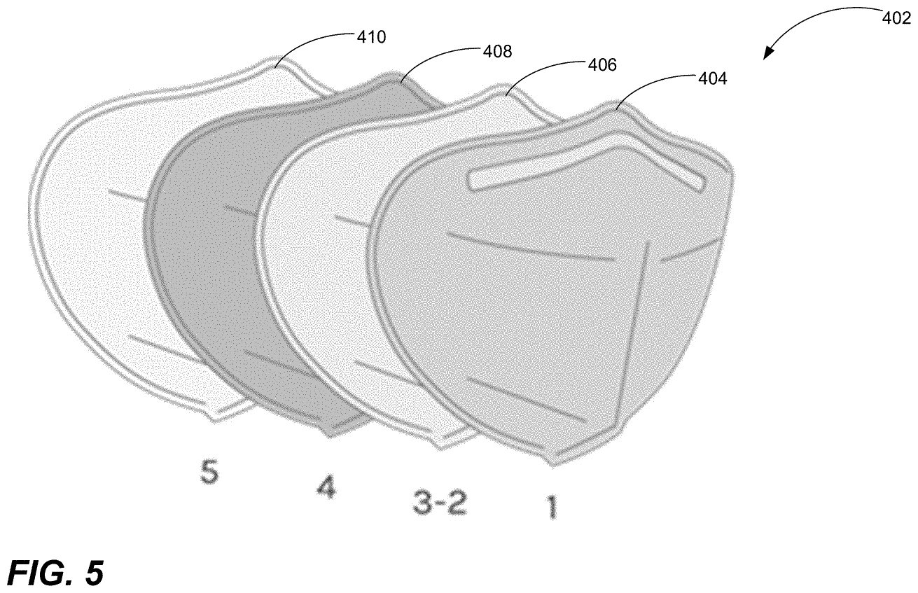

FIG. 5 is an illustration showing a face mask 402 with multiple layers 404-410, in accordance with some examples of the present disclosure In some examples, it may be desirable to construct a face mask using multiple layers, which are thereafter bonded to each other to form a final face mask, such as the face mask 402 of FIG. 5. Each of the layers 404-410 may be constructed of various materials and/or may serve to provide various functions. In some examples, various manufacturing processes using Polypropylene (“PP”), woven or non-woven, may be used to construct the layers having different mechanical properties. In some examples, the layers may be constructed from different types of spunbond nonwoven PP. As used herein, spun bond type nonwoven means the process of melting a polymer, and then spinning and drawing the melted polymer to produce filaments used to construct a nonwoven fabric. When drawing is realized in a powerful hot air flow, this results in a melt blown type nonwoven. It should be understood that the use of PP is merely an example, as other types of polymers may be used and are considered to be within the scope of the presently disclosed subject matter. In some examples, spunbond fibers can have a diameter of 10^{th} μm ensure the rigidity of the mask and filtration of large particles. Meltblown fibers may be thinner (a few micrometers) and are very efficient for the filtration of small particles. Meltblown fibers may be produced like spunbond, but a hot air stream contributes to stretch/draw the polymer jet resulting in thinner fibers.

For example, layer 404 may be the external (furthest from the user's face) layer that is used to provide structural support, some filtration, and/or protection to the face mask 402. Thus, the material or process used to construct the layer 404 may be such that the material has a greater stiffness or structural integrity than inner layers. The face mask 402 may also include one or more inner layers that provide additional functionality. For example, the layer 406 may be the layer having a viricidal and electrostatic charge applied to the layer 406, as described above. The layer 406 can provide additional filtration as well due to the mechanical structure of the PP used to construct the layer 406. In some examples, the layer 406 may be identified as the layer providing particle filtration and virus, bacteria or pathogen microorganisms neutralization. The face mask 402 may further include the layer 408 which may be used to provide additional particle filtration. The layer 408 may be used to filter larger particles. The mask 402 may further include layer 410, which may be used to provide a degree of comfort to the user, protection, and mechanical support to the face mask 402. It should be understood that various examples of the face mask 402 may include more than one of a particular type of layer, or, may not include one or more of the layers 404-410.

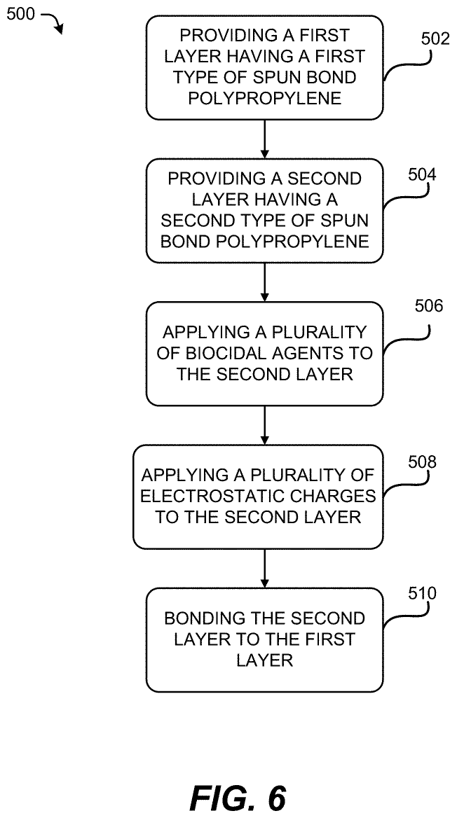

FIG. 6 is an example process 500 for manufacturing a face mask, such as the face mask 102 of FIG. 2 or 402 of FIG. 5, in accordance with some examples of the present disclosure. The process 500 and other processes described herein are illustrated as example flow graph. The order in which the operations are described is not intended to be construed as a limitation, and any number of the described operations can be omitted or combined in any order and/or in parallel to implement the processes.

The process 500 commences at operation 502, where a first layer having a first type of spunbond polypropylene nonwoven is provided. It should be noted that, although the description is provided for a spunbond polypropylene nonwoven, other types of nonwovens may be used, such as meltblown, and are considered to be within the scope of the presently disclosed subject matter. As used herein, a “type” is used to delineate differences in manufacturing, spinning, drawing, and the like used to create a certain layer using a melted polymer, and comprises melt blown and spun bond types. For example, the first layer may be used to provide some comfort as well as protection from projectiles droplets and mechanical support. The functionality of the first layer may be different than other layers, and thus, may be spun bond differently.

The process 500 continues to operation 504, where a second layer having a second type of spunbond polypropylene nonwoven is provided. In some examples, the second layer may provide different functions to the face mask than the first layer, and thus, may be spun bond differently than the first layer and other layers.

The process 500 continues to operation 506, where a plurality of biocidal agents is applied to the second layer. Examples of technologies used to apply the biocidal agents are described and referenced herein. In some examples, the plurality of biocidal agent comprises benzalkonium chloride complexed in cyclodextrin cavity bonded on fibers of the nonwoven material of the face mask. In some examples, the plurality of biocidal agents comprises fungicides, antimicrobials, viricidals, antibiotics, antibacterials, antivirals, antifungals, antiprotozoals, and antiparasites.

The process 500 continues to operation 508, where a plurality of electrostatic charges is applied to the second layer. In some examples, the electrostatic charges are applied directly after the application of the biocidal agents. In further examples, the electrostatic charges are applied using a high voltage potential or corona.

The process 500 continues to operation 510, where the second layer is bonded to the first layer. In some examples, the process 500 continues whereby additional layers having different spunbond nonwoven types are provided and bonded to other layers. The process thereafter ends.

As noted previously, various examples of the presently disclosed subject matter may be used in applications other than masks. For example, a gown, garment, covering of an object, or other uses may utilize the “catch and kill” properties of a layer of material using electrostatic charges to help capture particles and/or pathogen microorganisms and a biocidal agent to destroy or render inert the particle and/or pathogen microorganism. As mentioned above, in some examples, merely capturing an unwanted pathogen microorganism may have unintended consequences. For example, in a hospital setting, if gowns worn by medical practitioners merely capture unwanted organisms, when thrown away, the gowns, with viable pathogen microorganism, become a potential point source of contamination. This is especially concerning when a relatively high number of gowns are disposed of in a single container. The potential for contamination may be increased significantly because the potentially harmful pathogen microorganisms are now concentrated. On the other hand, if the gowns in the prior example merely have biocidal agents on the gown, without an electrostatic charge, the probability that the pathogen microorganism comes in contact with the biocidal agent is relatively lower. This is further exacerbated by the fact that the biocidal agent, in some cases, does not kill or render inert the pathogen microorganism merely upon contact. Often, there is a time of contact that is required for the biocidal agent to act on the pathogen microorganism. An electrostatic force, in some examples, not only increases the probability that the pathogen microorganism is captured, but can increase the time of contact between the pathogen microorganism and the biocidal agent due to electrostatic attraction, thus increasing the probability that the biocidal agent has sufficient time to kill or render inert the pathogen microorganism.

Thus, various examples of the presently disclosed subject matter may be used to produce cloth, fabric, or layers for non-mask applications. A suitable polymer may be used to manufacture a layer of fabric according to the various examples provided above. A biocidal agent may be affixed to fibers of the fabric and an electrostatic charge may be imparted on the fabric.

The presently disclosed examples are considered in all respects to be illustrative and not restrictive. The scope of the disclosure is indicated by the appended claims, rather than the foregoing description, and all changes that come within the meaning and range of equivalents thereof are intended to be embraced therein.

1. A face mask comprising:
a plurality of biocidal agents deposited on a first surface or a second surface of a material used to construct the face mask;
a plurality of electrostatic charges deposited using a high voltage source on the first surface or the second surface of the material used to construct the face mask; and
wherein the plurality of electrostatic charges increases a probability that a pathogen microorganism is captured by the first surface or the second surface and the plurality of biocidal agents kill or render inert the pathogen microorganism that is captured.

2. The face mask of claim 1, wherein the plurality of electrostatic charges is configured to increase an efficacy of a material used to construct the face mask to capture the pathogen microorganism.

3. The face mask of claim 1, wherein the face mask comprises an N95 respirator, a KN95 respirator, a bandana-type face mask, a pull-up face mask, a gaiter mask, a cloth mask, a European standard FFP2 Mask, an Australian P2 Mask, Korean 1^{st} class Mask, Japanese DS Mask, and a surgical mask.

4. The face mask of claim 1, wherein the plurality of biocidal agents comprises benzalkonium chloride complexed in cyclodextrin cavity bonded on fibers of the material of the face mask.

5. The face mask of claim 1, wherein the plurality of biocidal agents comprises antimicrobials, viricidals, antibiotics, antibacterials, antivirals, antifungals, antiprotozoals, and antiparasites.

6. The face mask of claim 1, wherein the biocidal agents comprise Dimethyldioctadecylammonium chloride.

7. The face mask of claim 1, wherein the biocidal agents comprise alkyldimethylbenzylammonium chloride.

8. The face mask of claim 1, wherein the biocidal agents comprise Benzethonium chloride.

9. The face mask of claim 1, wherein the face mask comprises a plurality of zones, wherein a concentration of the plurality of the biocidal agents and a concentration of the plurality of electrostatic charges varies between zones of the plurality of zones.

10. The face mask of claim 1, wherein a first zone of a plurality of zones inline with a direction of respirated air from a mouth of a wearer of the face mask has a higher concentration of either of the plurality of the biocidal agents or the plurality of the electrostatic charges than a second zone of a plurality of zones proximate to a side of a face of the wearer of the face mask.

11. A method of manufacturing a face mask, the method comprising:
providing a first layer having a first type of polypropylene nonwoven;
providing a second layer having a second type of polypropylene nonwoven;
applying a plurality of biocidal agents to the second layer;
applying a high voltage field to the second layer to deposit a plurality of electrostatic charges on the second layer; and
bonding the second layer to the first layer, wherein the plurality of electrostatic charges increases a probability that a plurality of pathogen microorganisms are captured by a surface of the second layer and the plurality of biocidal agents kill or render inert at least a portion of the plurality of pathogen microorganisms that are captured at the surface of the second layer.

12. The method of claim 11, further comprising:
providing a third layer having a first type of polypropylene nonwoven;
applying a plurality of biocidal agents to the third layer;
applying a high voltage field to the third layer to deposit a plurality of electrostatic charges on the third layer; and
bonding the third layer to the second layer, wherein the plurality of electrostatic charges increases a probability that at least a portion of the plurality of pathogen microorganisms are captured by a surface of the third layer and the plurality of biocidal agents kill or render inert at least a portion of the plurality of pathogen microorganisms that are captured at the surface of the third layer.

13. The method of claim 11, further comprising:
providing a third layer having a third type of polypropylene nonwoven;
applying a plurality of biocidal agents to the third layer; and
bonding the third layer to the second layer.

14. The method of claim 11, further comprising:
providing a third layer having a third type of polypropylene nonwoven;
applying a high voltage field to the third layer to deposit a plurality of electrostatic charges on the third layer; and
bonding the third layer to the second layer.

15. The method of claim 14, further comprising:
providing a fourth layer having a fourth type of polypropylene nonwoven; and
bonding the fourth layer to the third layer.

16. The method of claim 11, wherein the face mask comprises an N95 respirator, a KN95 respirator, a bandana-type face mask, a pull-up face mask, a gaiter mask, a cloth mask, a European standard FFP2 Mask, and a surgical mask.

17. The method of claim 11, wherein the plurality of biocidal agent comprises benzalkonium chloride complexed in cyclodextrin cavity bonded on fibers of a material of the face mask.

18. The method of claim 11, wherein the plurality of biocidal agents comprises fungicides, antimicrobials, viricidals, antibiotics, antibacterials, antivirals, antifungals, antiprotozoals, antiparasites, and combinations thereof.

19. The method of claim 11, wherein the biocidal agents comprise at least one of Dimethyldioctadecylammonium chloride, alkyldimethylbenzylammonium chloride, and Benzethonium chloride.

20. The method of claim 11, wherein applying a plurality of the biocidal agents to the second layer comprises:
identifying a first zone for a first density of the plurality of biocidal agents;
identifying a second zone for a second density of the plurality of biocidal agents; and
depositing the plurality of biocidal agents in the first zone at the first density and the second zone at the second density.

21. The method of claim 11, wherein applying a high voltage field to the second layer comprises:
identifying a first zone for a first density of the plurality of electrostatic charges; and
identifying a second zone for a second density of the plurality of electrostatic charges; and
applying the high voltage field at a first potential to deposit the plurality of electrostatic charges in the first zone at the first density and at a second potential to deposit the plurality of electrostatic charges in the second zone at the second density.

22. A face mask, comprising:
a first layer having a first type of polypropylene nonwoven;
a second layer having a second type of polypropylene nonwoven;
a plurality of biocidal agents on the second layer;
a plurality of electrostatic charges on the second layer deposited using a high voltage field applied to the second layer to deposit the plurality of electrostatic charges; and
the second layer bonded to the first layer, wherein the plurality of electrostatic charges increases a probability that a plurality of pathogen microorganisms are captured by a surface of the second layer and the plurality of biocidal agents kill or render inert at least a portion of the plurality of pathogen microorganisms that are captured at the surface of the second layer.

23. The face mask of claim 22, further comprising:
a third layer having a first type of polypropylene nonwoven;
a plurality of biocidal agents on the third layer;
a plurality of electrostatic charges on the third layer deposited using a high voltage field applied to the third layer to deposit the plurality of electrostatic charges; and
the third layer bonded to the second layer, wherein the plurality of electrostatic charges increases a probability that at least a portion of the plurality of pathogen microorganisms are captured by a surface of the third layer and the plurality of biocidal agents kill or render inert the at least the portion of the plurality of pathogen microorganisms that are captured at the surface of the third layer.

24. The face mask of claim 22, further comprising:
a third layer having a third type of polypropylene nonwoven;
a plurality of biocidal agents to the third layer; and
the third layer bonded to the second layer.

25. The face mask of claim 22, further comprising:
a third layer having a third type of polypropylene nonwoven;
a plurality of electrostatic charges on the third layer deposited by applying a high voltage field to the third layer; and
the third layer bonded to the second layer.

26. The face mask of claim 25, further comprising:
a fourth layer having a fourth type of polypropylene nonwoven; and
the fourth layer bonded to the third layer.

27. The face mask of claim 22, wherein the face mask comprises an N95 respirator, a KN95 respirator, a bandana-type face mask, a pull-up face mask, a gaiter mask, a cloth mask, a European standard FFP2 Mask, and a surgical mask.

28. The face mask of claim 22, wherein the plurality of biocidal agent comprises benzalkonium chloride complexed in cyclodextrin cavity bonded on fibers of a material of the face mask.

29. The face mask of claim 22, wherein the plurality of biocidal agents comprises fungicides, antimicrobials, viricidals, antibiotics, antibacterials, antivirals, antifungals, antiprotozoals, antiparasites, and combinations thereof.

30. The face mask of claim 22, wherein the plurality of biocidal agents comprises at least one of Dimethyldioctadecylammonium chloride, alkyldimethylbenzylammonium chloride, and Benzethonium chloride.

31. A fabric, comprising:
a layer having a type of polypropylene nonwoven;
a plurality of biocidal agents on the layer;
a plurality of electrostatic charges on the layer deposited using a high voltage field applied to the layer to deposit the plurality of electrostatic charges; and
wherein the plurality of electrostatic charges increases a probability that a plurality of pathogen microorganisms are captured by a surface of the layer and the plurality of biocidal agents kill or render inert at least a portion of the plurality of pathogen microorganisms that are captured at the surface of the layer.

32. The fabric of claim 31, further comprising:
a second layer having a first type of polypropylene nonwoven;
a plurality of biocidal agents on the second layer;
a plurality of electrostatic charges on the second layer deposited using a high voltage field applied to the second layer to deposit the plurality of electrostatic charges; and
the second layer bonded to the layer, wherein the plurality of electrostatic charges increases a probability that at least a portion of the plurality of pathogen microorganisms are captured by a surface of the second layer or the layer and the plurality of biocidal agents kill or render inert the at least a portion of the plurality of pathogen microorganisms that are captured at the second layer.

33. The fabric of claim 32, further comprising:
a third layer having a third type of polypropylene nonwoven;
a plurality of biocidal agents to the third layer; and
the third layer bonded to the second layer.

34. The fabric of claim 32, further comprising:
a third layer having a third type of polypropylene nonwoven;
a plurality of electrostatic charges on the third layer deposited by applying a high voltage field to the third layer; and
the third layer bonded to the second layer.

35. The fabric of claim 34, further comprising:
a fourth layer having a fourth type of polypropylene nonwoven; and
the fourth layer bonded to the third layer.

36. (canceled)

37. The fabric of claim 31, wherein the plurality of biocidal agent comprises benzalkonium chloride complexed in cyclodextrin cavity bonded on fabric.

38. The fabric of claim 31, wherein the plurality of biocidal agents comprises fungicides, antimicrobials, viricidals, antibiotics, antibacterials, antivirals, antifungals, antiprotozoals, antiparasites, and combinations thereof.

39. The fabric of claim 31, wherein the plurality of biocidal agents comprises at least one of Dimethyldioctadecylammonium chloride, alkyldimethylbenzylammonium chloride, and Benzethonium chloride.
